(19) United States
(12) Patent Application Publication
(10) Pub. No.: US 2022/0068458 A1
(43) Pub. Date: Mar. 3, 2022

(54) ARTIFICIAL INTELLIGENCE-BASED ALGORITHM FOR PHYSIOTHERAPY AND REHABILITATION ROBOTS FOR DIAGNOSIS AND TREATMENT PURPOSES

(71) Applicants: YILDIZ TEKNIK ÜNIVERSITESI, Esenler/lstanbul (TR); BARTIN ÜNIVERSITESI, Bartin (TR)

(72) Inventors: Erhan AKDOGAN, Istanbul (TR); Mehmet Emin AKTAN, Bartin (TR)

(21) Appl. No.: 17/415,791

(22) PCT Filed: Dec. 10, 2019
(86) PCT No.: PCT/TR2019/051053

§ 371 Date: Jun. 18, 2021

(30) Foreign Application Priority Data
Dec. 18, 2018 (TR) 2018/19746

(51) Int. Cl. G16H 20/30; G16H 10/60; G16H 50/20

(52) U.S. Cl. CPC G16H 20/30; G16H 50/20; G16H 10/60

(57) ABSTRACT

The invention is an artificial intelligence-based algorithm which enables physiotherapy and rehabilitation robots to perform diagnosis and treatment by using biomechanical measurements and which comprises central processing unit (3) which is a management unit identifying joint range of motion and/or strength/torque deficiency by using information that it receives from other perimeter units contained in the system and from the robot (2) controlled by this algorithm.

### TECHNICAL FIELD

The invention relates to an artificial intelligence-based algorithm which is developed for physiotherapy and rehabilitation robots to perform both diagnosis and treatment.

### BACKGROUND ART

The systems that are used in robotic rehabilitation are substantially used for treatment purposes. In conventional diagnosis method, which is conducted in clinics, joint range of motion of a patient is measured by a miter (goniometer) and clamping force is measured by a dynamometer. Joint force and torques are not measured. At diagnosis phase one of the difficulties which is experienced by doctors is that biomechanical parameters (joint range of motion and joint force/torques) are not completely measured. This is a problem in terms of recording the treatment process and following the development of the patient. Furthermore, patients are scheduled appointments for days later due to the limited daily number of patients to be accepted by doctors and density of population. Long and costly treatment, difficulty of access by patients to treatment centers, the fact that physiotherapists cannot enable the same conditions in exercises including repetitive motions to be applied manually may be among other problems.

Considering that artificial intelligence-based technologies are used in every field of our life, the presence of an artificial intelligence-based system which is developed for diagnosis and treatment purposes in physiotherapy and rehabilitation will contribute to technological development and relieve the workload of doctors and physiotherapists. There are some robots which are developed for diagnosis in physiotherapy in the prior art.

In Chinese patent application no. CN106806996 titled “Diagnosis and treatment integrated laser physiotherapy intelligent robot and control method”, the invention is an integrated laser physiotherapy intelligent robot for diagnosis and treatment comprising a laser treatment module, a robot module, an auxiliary analysis module and a central control module, and the robot module includes an interconnected robot arm unit, an arm five-axes movement unite and a robot control unit. The medical diagnosis system stated in the invention is comprised of a medical infra-red thermal imaging system, a heat graphic imaging system and analysis software. Infra-red thermal imaging device may take a partial picture of human body, creates a heat-area picture, display it along the screen and can find hot point or abnormal inflammation hot area of human body through integrated analysis software.

The invention in a Chinese patent no. CN108091392 entitled “A diagnosis and treatment-integrated physiotherapy device” is an integrated physiotherapy device for diagnosis and treatment and including the main control system which is structured to analyse human body tissue image and to form a diagnosis result and treatment plan, an image capturing system for collecting tissue images of human body and a physical factor output control system in order to produce a physiotherapy factor corresponding to a treatment plan and activating the same on human tissue, in which the system transfers the data. The image capturing system comprises a visible light image capturing device and an infrared image capturing device.

### BRIEF DESCRIPTION OF THE INVENTION

An intelligent control structure based on an artificial intelligence which works in an information and rule-based manner, in which statistical techniques are used for determining the degrees of deficiency in force and joint range of motion in limbs and to determine exercise methods and parameters as an alternative to conventional method of diagnosis in clinics is developed in the invention. Joint range of motion and joint force values are measured completely and in precise accuracy through this structure. According to the measurements conducted and to physical and personal characteristics of a patient; deficiencies in range of motion and force values are determined by means of healthy human database having eigen value, correlation analysis unit, regression analysis unit and biomechanical parameter extraction unit. Exercise types and exercise parameters are determined by means of therapeutic exercise database which is developed originally using those deficiencies and patient biomechanical measurements. This determined exercise information are sent to specialists via a mobile application. Therefore, the system performs all measurement and determining processes.

The inventive method shall be able to be applied to novel and currently available robotic systems which are developed in lower and upper extremity rehabilitation.

### DESCRIPTION OF THE INVENTION

The aim of invention is to form an artificial intelligence-based algorithm which performs diagnosis in physiotherapy and rehabilitation and which uses biomechanical parameters in order to determine exercise methods and parameters, in which statistical techniques are used. Another aim of invention in line with the foregoing aim is to create a mobile application in order to send the determined exercise information to specialists.

### DESCRIPTION OF FIGURES

FIG. 1: Block diagram of the system

### DEFINITION OF REFERENCE SIGNS

1. User information

2. Robot

3. Central processing unit

4. Healthy human database

5. Correlation analysis unit

6. Regression analysis unit

7. Biomechanical parameter extraction unit

8. Therapeutic exercise database

9. Conventional controller

10. Mobile application

11. Cloud database

The invention is an artificial intelligence-based system which works in an information and rule-based manner that enables measuring biomechanical parameters, in which statistical techniques are used to perform diagnosis in physiotherapy and rehabilitation and to determine exercise methods and parameters, wherein it comprises a central processing unit (3), a robot (2), a user unit (1), a healthy human database (4), a correlation analysis (5), a regression analysis (6), a biomechanical parameter extraction unit (7), a therapeutic exercise database (8), a conventional controller (9), a mobile application (10) and cloud database (11) units.

The user unit (1) is the unit where data such as name-surname, sex, age, height, weight, extremity sizes of a patient is entered by the system user. Robot (2) unit is the unit performing biomechanical measurements of patients. Biomechanical measurements here are clamping force, flexion joint range of motion and joint strength, extension joint range of motion and joint strength, ulnar deviation joint range of motion and joint strength, radial deviation joint range of motion and joint strength, pronation joint range of motion and joint torque, supination joint range of motion and joint torque.

Central processing unit (3) is the management unit enabling data communication in the system, diagnosing joint range of motion and strength/torque deficiency by using the information which it receives from system biomechanical parameter extraction unit (7). Healthy human database (4) is the database including biomechanical parameters of healthy human whose data are entered to system in order for the system to perform diagnosis process.

Correlation analysis unit (5) is the unit which determines the independent variables affecting on dependent variables in healthy human database (4) and which sends the same to regression analysis unit (6). As a consequence of the correlation analysis performed in this unit, independent variables having high and very high correlation on dependent variables are determined. For example, a 0.8 correlation factor between flexion strength and arm perimeter measurement shows a high correlation inbetween. In this situation, arm perimeter, which is the independent variable, is sent to regression analysis unit (6).

Regression analysis unit (6) is the unit which forms the factor matrix. The number of lines is related to the movement types and the number of columns is related to the number of independent variables. The unit calculates the factor matrix which is formed of partial regression factors forming the relation between the selected variables that are sent by correlation analysis unit (5) and six dependent variable and send the matrix to biomechanical parameter extraction unit (7). The number of independent variables is determined by the correlation analysis unit (5) based on the variables with high correlation.

Biomechanical parameter extraction unit (7) is the unit that determines the required biomechanical parameter values by using the physical characteristics of a patient received from central processing unit (3) and factor matrix received from regression analysis unit (6).

Therapeutic exercise database (8) is the unit comprising joint range of motion and strength values which are required to be applied in each set with exercise types and the information of the situations in which those exercises are used. The role of this unit is to determine exercise type and parameters based on deficiency percentages and patient biomechanical measurements. For example, passive exercise with 80° joint range of motion may be suggested to a patient with 100% joint range of motion deficiency in flexion direction.

Conventional controller (9) is the unit which selects the appropriate controller based on exercise type and parameters received from central processing unit (3) and sends the same to motor drivers by calculating the necessary torque values.

Cloud database (11) is the database which may be accessed over internet in which all data in the said units of the system are kept.

Mobile application (10) performs the communication between patient, specialist and robot.

Biometrical measurements such as patient information that is formed by profile and/or physical information entered to user unit (1) by the system user and position & strength data made by robot (2) manipulator are transferred to biomechanical measurements central processing unit (3) in the flow of data between the units of the inventive system. Healthy human data are entered into healthy human database (4) over central processing unit (3).

Dependent variables in the healthy human database (4) are sent to correlation analysis unit (5), independent variables which are effective over dependent variables are determined by the correlation analysis performed in this unit. The determined independent variables are sent to regression analysis unit (6).

Regression analysis unit (6) is the unit which forms the factor matrix. The unit calculates the factor matrix which is formed of partial regression factors forming the relation between the selected variables that are sent by correlation analysis unit (5) and six dependent variable and send the matrix to biomechanical parameter extraction unit (7).

Biomechanical parameter extraction unit (7) is the unit that determines the required biomechanical parameters by using the physical characteristics of a patient received from central processing unit (3) and factor matrix received from regression analysis unit (6).

Central processing unit (3) sends patient information that is entered by the user (sex, age, height and weight, extremity sizes etc.) to biomechanical parameter extraction unit (7) and receives the required joint range of motion and strength/torque information received from biomechanical parameter extraction unit (7).

Central processing unit (3) extracts the values of the patient from the required joint range of motion and strength/torque values and determines joint range of motion and strength/torque deficiency percentages. The unit sends the strength & JRM deficiency percentages and biomechanical measurements to therapeutic exercise database (8), receives exercise type and parameters which are required to be applied and sends them to specialists via mobile application (10) over cloud database (11) in combination with the exercise tips.

Specialists carry out the necessary examinations and may send approval, rejection or correction about exercise tips to central processing unit (3) via mobile application (10). Central processing unit (3) transfers to conventional controller (9) the rejected or corrected exercise information that is received. Central processor (3) selects the appropriate control method present in the conventional controller (9), calculates the necessary motor torque value and sends it to the motor drivers.

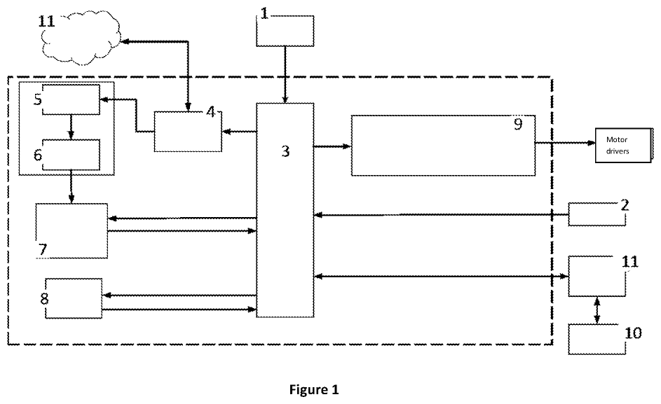

The inventive system comprises receiving patient information from the user and entering the same to the central processing unit (3), measuring the biomechanical parameters of the patient via robot (2) (position and strength data) and entering the same to the central processing unit (3), sending the patient information (sex, age, height and weight, extremity sizes) to parameter extraction unit (7), sending the information present in the healthy human database (4) to correlation analysis unit (5) and determining independent variables which are effective over dependent variables by performing correlation analysis, sending the independent variables to regression analysis unit (6), and sending the same to biomechanical parameter extraction unit (7) by performing regression analysis and determining equation factors, determining the biomechanical values to be present under normal conditions by using equation and patient information by biomechanical parameter extraction unit (7) and sending the same to the central processing unit (3), comparing the measurements performed by the robot (2) and values that are received from biomechanical parameter extraction unit (7) in central processing unit (3) and determining the deficiency percentages of patient biomechanical parameters for determining the biomechanical values in order to determine strength & JRM deficiency percentages and biomechanical measurements in accordance with the biomechanical values which should be present in the patient under normal conditions as shown in the block diagram of FIG. 1.

The inventive system comprises the steps which include sending biomechanical measurements and deficiency percentages of the patient to therapeutic exercise database (8) by the central processing unit (3), determining the exercises to be applied based on the deficiency percentages and exercise parameters based on patient measurements by the therapeutic exercise database (8) and sending the same to the central processing unit (3), sending the exercise method and parameters to a specialist optionally by central processing unit (3) via a mobile application (10), sending the motor control signs in accordance with exercise method and parameters by central processing unit (3) to the robot (2) via conventional controller (9), and sending the motor control signs in accordance with exercise method and parameters by the central processing unit (3) via conventional controller (9) to the robot (2) in order to determine the therapeutic exercise types and parameters in accordance with the diagnosed strength and JRM deficiency percentages and biomechanical measurements of the patient and transferring them to the specialists.

### DETAILED DESCRIPTION OF THE INVENTION

The invention is an artificial intelligence-based algorithm which enables physiotherapy and rehabilitation robots to perform diagnosis and treatment by using biomechanical measurements and which comprises central processing unit (3) which is a management unit identifying joint range of motion and/or strength/torque deficiency by using information that it receives from other perimeter units contained in the system and from the robot (2) controlled by this algorithm.

In the invention, the profile information and/or physical information is entered to the system over the user unit (1) by the system user.

A healthy human database (4) is present in order to enter biomechanical parameters of healthy human, which are used for performing determination operation of the system.

The inventive system comprises correlation analysis unit (5) which determines independent variables that are efficient over the dependent variables in the healthy human database (4) and regression analysis unit (6) which calculate factor matrix formed of partial regression factors which form the relation between the selected independent variables that are sent by the correlation analysis unit (5) and six dependent variables.

Biomechanical parameter extraction unit (7) determines the required biomechanical parameters by using factor matrix received from regression analysis unit (6) and physical characteristics of patient received from central processing unit (3) to which factor matrix calculated by the regression analysis unit (6) mentioned in the system.

In the present invention, the central processing unit (3) has inter-database flow in order to determine exercise methods and parameters besides the determination of joint range of motion and/or strength/torque deficiency.

Therapeutic exercise database (8) is the unit comprising joint range of motion and strength values which are required to be applied in each set with exercise types and the information of the situations in which those exercises are used. The role of this unit is to determine exercise type and parameters based on deficiency percentages and patient biomechanical measurements.

Conventional controller (9) is the unit which selects the appropriate controller based on exercise type and parameters received from central processing unit (3) and sends the same to motor drivers by calculating the necessary torque values.

In the present invention, there is cloud database (11) functioning as the database which is accessed over internet, where all data in the mentioned units of the system are contained.

The communication between patient, specialist and robot is performed via mobile application (10) in the inventive algorithm.

Biometrical measurements such as patient information entered to user unit (1) by the system user and position & strength data made by robot (2) manipulator are transferred to biomechanical measurements central processing unit (3) in the flow of data between the units of the inventive system. Healthy human data are entered into healthy human database (4) over central processing unit (3).

Dependent variables in the healthy human database (4) are sent to correlation analysis unit (5) and independent variables which are effective over dependent variables are determined by the correlation analysis performed in this unit. The determined independent variables are sent to regression analysis unit (6).

Regression analysis unit (6) is the unit which forms the factor matrix. The unit calculates the factor matrix which is formed of partial regression factors forming the relation between the selected variables that are sent by correlation analysis unit (5) and six dependent variable by a programming language and sends the matrix to biomechanical parameter extraction unit (7).

Biomechanical parameter extraction unit (7) uses patient information (sex, age, height and weight, extremity size etc.) received from the central processing unit (3) and factor matrix received from regression analysis unit (6), determines desired strength & JRM deficiency percentages and biomechanical measurements and sends the same to the central processing unit (3).

The central processing unit (3) extracts the biomechanical measurement values that are measured by the robot (2) from the required biometrical parameter values received from the biomechanical parameter extraction unit (7), thus determines deficiency percentages of the biomechanical parameters.

It sends the determined strength & JRM deficiency percentages and biomechanical measurements to therapeutic exercise database (8). Therapeutic exercise database (8) determines the required exercise types and parameters and sends the same to the central processing unit (3). The central processing unit (3) sends exercise tips and patient information to specialist or specialists via mobile application (10) over a cloud database (11).

Specialists examines the exercise types and parameter tips together with patient information and may send approval, rejection or correction about exercise tips to central processing unit (3) via mobile application (10). Central processing unit (3) transfers to conventional controller (9) the accepted or corrected exercise information that is received. Central processor (3) selects the appropriate control method present in the conventional controller (9) in accordance with the approval or corrections, calculates the necessary motor torque value and sends it to the motor drivers. Conventional controller (9) sends motor control signs to robot (2) in accordance with exercise management and parameters.

1. An artificial intelligence-based algorithm for physiotherapy and rehabilitation robots for diagnosis and treatment purposes using biomechanical measurements of patients, wherein; it comprises a central processing unit which is the management unit carrying out data communication, determining joint range of motion and/or strength/torque deficiency using the information that it receives from the robot and other perimeter units in the system.

2. An artificial intelligence-based algorithm for physiotherapy and rehabilitation robots according to claim 1, wherein; it comprises a user unit (in which profile information and/or physical information of the patient is entered by the system user.

3. An artificial intelligence-based algorithm for physiotherapy and rehabilitation robots in accordance with claim 1, wherein; it comprises healthy human database where biomechanical parameters of healthy human whose data are entered to the system are contained in order for the system to carry out the diagnostic process.

4. An artificial intelligence-based algorithm for physiotherapy and rehabilitation robots in accordance with claim 3, wherein; it comprises a correlation analysis unit which determines the independent variables that are effective on the dependent variables in the healthy human database.

5. An artificial intelligence-based algorithm for physiotherapy and rehabilitation robots in accordance with claim 4, wherein; it comprises a regression analysis unit which calculates the factor matrix in which factors of the equations forming the mathematical relation between selected independent variables that are received from the correlation analysis unit and the dependent variable.

6. An artificial intelligence-based algorithm for physiotherapy and rehabilitation robots in accordance with claim 5, wherein; it comprises a biomechanical parameter extraction unit which determines the required biomechanical parameters by using factor matrix received from regression analysis unit and patient profile information and/or physical information received from central processing unit.

7. An artificial intelligence-based algorithm for physiotherapy and rehabilitation robots in accordance with claim 1, wherein; it comprises a therapeutic exercise database which determines exercise types and parameters in accordance with strength/torque & JRM deficiency percentages that are identified by the central processing unit and which contains exercise types and the information regarding the situations for performing the exercises.

8. An artificial intelligence-based algorithm for physiotherapy and rehabilitation robots in accordance with claim 7, wherein; it comprises a therapeutic exercise database including joint range of motion and strength values to be applied in each set.

9. An artificial intelligence-based algorithm for physiotherapy and rehabilitation robots in accordance with claim 7, wherein; it comprises a conventional controller which selects the appropriate controller in accordance with exercise type and parameters, calculates the necessary torque values and sends them to motor drivers.

10. An artificial intelligence-based algorithm for physiotherapy and rehabilitation robots in accordance with claim 1, wherein; it comprises a cloud database which may be accessed via internet, comprising all data in the mentioned units of the system.

11. An artificial intelligence-based algorithm for physiotherapy and rehabilitation robots in accordance with claim 1, wherein; it comprises mobile application in order to carry out the communication between patient, specialist and robot.

12. An artificial intelligence-based algorithm for physiotherapy and rehabilitation robots in accordance with claim 1, wherein it comprises the process steps of;
receiving the patient information that is entered to the user unit by the system user and biomechanical measurements concerning position and strength data carried out by robot manipulators by the central processing unit,
sending the dependent variables in the healthy human database to correlation analysis unit, by performing the correlation analysis and determining the independent variables that are effective on dependent variables and sending the same to the regression analysis unit,
calculating the factor matrix that is formed of partial regression factors constituting the relation between the selected independent variables and dependent variable and sending the same to the biomechanical parameter extraction unit by the regression analysis unit,
determining the desired strength/torque & JRM deficiency percentages and biomechanical measurements, which should be present under normal conditions by using the said patient information received by the central processing unit and factor matrix received from the regression analysis unit and sending of the same by the biomechanical parameter extraction unit to the central processing unit,
extracting the biomechanical measurement values of the patient that are measured by the robot from the required biometrical parameter values received from the biomechanical parameter extraction unit, thus determining deficiency percentages of the biomechanical parameters the central processing unit.

13. An artificial intelligence-based algorithm for physiotherapy and rehabilitation robots in accordance with claim 12, wherein it comprises the process steps of;
sending the strength & JRM deficiency percentages and biomechanical measurements determined by the central processing unit to therapeutic exercise database,
determining the exercise type which should be applied in accordance with the deficiency percentages and biomechanical measurements and determining the exercise parameters in accordance with the patient measurements in therapeutic exercise database,
sending the exercise types together with patient information via the mobile application over the cloud database to the specialist by receiving of the exercise type and parameters information to be carried out from the therapeutic exercise database by the central processing unit.

14. An artificial intelligence-based algorithm for physiotherapy and rehabilitation robots in accordance with claim 13, wherein; it comprises the process step of;
examining the exercise types and parameter types together with patient information and then sending approval, rejection or correction concerning those types to central processing unit via mobile application by the specialist,

15. An artificial intelligence-based algorithm for physiotherapy and rehabilitation robots in accordance with claim 14, wherein; it comprises the process steps of;
transferring the approved or corrected exercise information that is received from the specialist to the conventional controller by the central processing unit,
selecting the appropriate control method present in the conventional controller in accordance with the approval or corrections, calculating the necessary motor torque value and sending of the same to the motor drivers.

16. An artificial intelligence-based algorithm for physiotherapy and rehabilitation robots in accordance with claim 15, wherein; it comprises the process step of; sending the motor control signs in accordance with exercise method and parameters to the robot by the conventional controller.
